(19) United States
(12) Patent Application Publication
(10) Pub. No.: US 2004/0002625 A1
(43) Pub. Date: Jan. 1, 2004

(54) Apparatus and methods for cardiac surgery

(76) Inventors: Timothy Dietz, Califon, NJ (US); Daniel M. Brounstein, Fremont, CA (US); Meir Moshe, El Sobrante, CA (US); Jaime R. Gannoe, Palo Alto, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/183,920

(22) Filed: Jun. 27, 2002

(51) Int. Cl. A61F 2/00

(52) U.S. Cl. 600/37

(57) ABSTRACT

The invention provides a stabilizer having a suction foot to stabilize a patient's heart. One embodiment of the stabilizer has two suction feet, each having two chambers with different volumes. A shoulder having a suction manifold is formed with an inner surface of at least one chamber so as to enhance the preferential draw of medial tissue. The volumes of the chambers and the orifices and suction manifold are configured to produce vacuum independence between the chambers and between the suction feet.

### FIELD OF THE INVENTION

The present invention relates generally to surgical instruments, and more specifically to heart stabilizers that utilize a suction source to minimize motion of heart tissue during cardiac surgery.

### BACKGROUND OF THE INVENTION

In heart surgery, an incision is made in the chest, either through the sternum (a median sternotomy), between the ribs (a thoracotomy), or some combination of the two, to gain access into the chest cavity. A retractor is positioned within the chest incision to spread the chest bones and/or tissue to create an opening. Surgical instruments are then placed through this opening to perform surgery on the heart.

One of the most common types of heart surgery is coronary artery bypass grafting, or CABG. In CABG, a blockage in one or more coronary arteries is bypassed by connecting a graft vessel to the coronary artery downstream of the blockage. The technique of connecting the graft vessel to the coronary artery is known as anastomosis. The graft vessel may be a mammary artery dissected from the chest wall, where the upstream end of the artery is left intact and the downstream end is attached to the coronary artery. Alternatively, the graft vessel may be a section of artery or vein from elsewhere in the patient's body, or an artificial vascular graft, where the upstream end of the graft is attached to an artery such as the aorta, and the downstream end is connected to the coronary artery. In this way, multiple coronary artery blockages at various locations on the front, side or back of the heart may be bypassed using multiple graft vessels.

Conventionally, CABG is performed with the heart stopped, while the patient is supported on cardiopulmonary bypass, whereby the patient's blood is circulated by means of an extracorporeal pump and oxygenation system. In certain cases, however, CABG may be performed with the heart beating in a technique known as “beating-heart” or “offpump” coronary artery bypass (OPCAB), thereby obviating the need for cardiopulmonary bypass. In OPCAB, the surface of the heart near the anastomosis site on the coronary artery is stabilized using a specialized instrument while the heart continues to beat. This local stabilization helps to minimize motion at the anastomosis while the graft vessel is being connected by the surgeon to the coronary artery. Typically, the coronary artery is temporarily occluded or a temporary shunt is inserted into the coronary artery during the anastomosis procedure to keep the site free of blood.

Currently, there are several devices being sold as heart stabilizers. Generally, the devices minimize the relative movement of tissue contacted by and near the stabilizer by applying a force to the heart surface to constrain at least a portion of the heart from beating and moving as it would absent the force. These prior art stabilizers often use a vacuum source to assist in capturing the heart tissue that contacts the stabilizer and to better expose the target coronary artery for surgery. Examples of prior art devices include the Octopus suction stabilizer (Medtronic) and the Axius mechanical stabilizer (Guidant) and Vortex suction stabilizer (Guidant). Further examples of prior art stabilizers are disclosed in part PCT International Publication No. WO 00/15119 (Borst), U.S. Pat. Nos. 6,036,641 (Taylor), 6,394,951 (Taylor) and 6,032,672 (Benetti), and copending U.S. patent application Ser. No. 09/492,558 (assigned to the assignee of the present application), each of which is incorporated herein by reference.

### SUMMARY OF THE INVENTION

The present invention provides a devices and methods for stabilizing a predetermined area on a heart or other organ of a patient to permit a surgical procedure, such as OPCAB, to be performed.

One preferred embodiment of the stabilizer of the current invention includes a shaft attached on one end to a mounting assembly and on the other end to a suction assembly. The suction assembly is configured to engage the surface of the heart on opposing sides of an anastomosis site, and includes a frame that preferably is pivotably mounted to the distal end of the shaft by an articulating joint, a first suction foot and a second suction foot, connected one to the other by the frame. The suction feet each include a housing that is formed with at least two chambers, wherein at least two of the chambers have different volumes. The housing further includes a shoulder molded into an under surface along the length of the proximal-most chamber. The shoulder has a manifold that is in fluid communication with a suction inlet port, which in turn communicates with a suction source. The shoulder also has a distal orifice that permits fluid communication between the manifold and the distal chamber, and a proximal orifice that permits fluid communication between the manifold and the proximal chamber.

In the preferred embodiment, the proximal and distal orifices, the manifold cross section and the volume of each of the chambers are configured such that the chambers have vacuum independence. For purposes of this application “vacuum independence” means that the vacuum produced in each of the chambers is such that, if suction is lost in one chamber such that it no longer holds cardiac tissue, the other chamber or chambers has sufficient vacuum to maintain hold of cardiac tissue. In other words, the loss of vacuum in one chamber does not cause the remaining chamber or chambers to also lose its hold on cardiac tissue.

In one embodiment, the suction feet have two chambers, a distal chamber that has a first volume and a proximal chamber that has a second volume that is greater than the distal chamber first volume. The difference in volumes between the two chambers cause the distal chamber to be evacuated first, thereby producing a vacuum within the distal chamber that will capture tissue prior to the proximal chamber. In this way, a practitioner may grossly position the foot by positioning the distal end of the foot first and then finetune the position of the remainder of the foot. The chambers of the feet develop sufficient capture vacuums at different rates due to the different volumes and orifice sizes, and as such capture tissue at different rates.

The suction feet may be designed to pull taut the medial tissue to better expose the coronary or target artery for purposes of performing CABG. The medial tissue is that tissue located between the first foot and the second foot once the suction assembly has been positioned on the heart. The medial edge of at least one chamber of the suction feet are configured to preferentially permit medial tissue to be drawn or pulled into the chamber. To create an edge that permits the medial tissue to move relative to the edge, the medial edge of the chambers includes a means to reduce friction relative to the medial tissue. The friction-reducing means includes rounding or chamfering the medial edge or the application of a hydrophilic coating to the medial edge, such as silicone, or covering the medial edge with a friction-reducing material or modifying the medial edge surface to make it smoother, such as by a plasma treatment, or any combination of any of the foregoing.

In addition, to enhance the preferential drawing of medial tissue, the lateral edge of the chamber or chambers may include a means to increase friction relative to the lateral tissue to resist lateral tissue from being drawn into the chambers from the lateral side. The lateral edge of the chambers is the edge adjacent the lateral tissue, which lies outside the medial tissue. The friction-increasing means includes squaring off the lateral edge, or the lateral edge may be textured, knurled, roughened, or covered or coated with a friction-enhancing material or any combination of any of the foregoing. In this way, more tissue on the medial side of each foot is drawn into the chamber than on the lateral side, thereby causing the medial tissue to be pulled taut to better expose the target artery for CABG. For purposes of this application, the lateral side of a foot is that side closer to lateral tissue, and the medial side of a foot is that side closer to medial tissue.

To further enhance the preferential draw of medial tissue, the ceiling or inner surface of one or more of the chambers may included a shoulder that is molded closer to the lateral edge such that when lateral tissue has been pulled into the chamber, the lateral tissue contacts the shoulder thereby providing further resistance to tissue flow from the lateral side. The shoulder, in effect, increases the friction encountered by the lateral tissue, as the tissue is subject to two contact points on the lateral side, the lateral edge and the shoulder, as compared to the one contact point on the medial side, the medial edge. Further, once the lateral tissue contacts the shoulder, it isolates the region above the lateral tissue from the vacuum source thereby reducing the area of lateral tissue acted upon by the vacuum within the chambers.

To enhance the practitioner's ability to position the feet on the irregular surface of the heart, the frame may be configured to be bendable in each of the three degrees of freedom. The frame may include at least two living hinges that permit the frame to bend at each point and take a set to maintain the suction feet at a preferred position.

To provide access through small ports, such as through an intercostal space, the stabilizer may include suction tubing having coil-reinforced polyurethane tubing to provide a suction source without concern that it will become kinked as it is passed through relatively small openings.

A further understanding of the nature and advantages of the invention and further aspects and advantages of the invention may be realized by reference to the remaining portion of the specification and the drawings.

### BRIEF DESCRIPTION OF THE DRAWINGS

FIG. 1 is a perspective view of a retraction and stabilization system according to the invention.

FIG. 2 is a bottom perspective view of the stabilizer suction feet of FIG. 1.

FIG. 3 is a top perspective view of the feet of FIG. 1.

FIG. 4 is a bottom plan view of a foot of FIG. 1.

FIG. 5 is a side cross-sectional view of the foot of FIG. 4.

FIGS. 6A-6C are cross-sectional views, taken along line 6A-6A of FIG. 4, of the feet at different stages of engagement with cardiac tissue.

FIG. 7 is a cross-sectional view of the feet of FIG. 1 at different stages of engagement with cardiac tissue.

FIG. 8 is a detailed cross-sectional view of one embodiment of the lateral and medial edges of the stabilizer feet of FIG. 1.

FIG. 9 is a top plan view of a second embodiment of a stabilizer suction foot.

FIG. 10 is a cross-sectional view of the stabilizer foot of FIG. 9.

FIG. 11 is a bottom plan view of the stabilizer foot of FIG. 9.

### DETAILED DESCRIPTION OF THE SPECIFIC EMBODIMENTS

Although this invention is applicable to numerous and various types of organs and surgical procedures, it has been found particularly useful in the environment of surgical procedures on the heart. Therefore, without limiting the applicability of the invention to surgical procedures on the heart, the invention will be described in such environment.

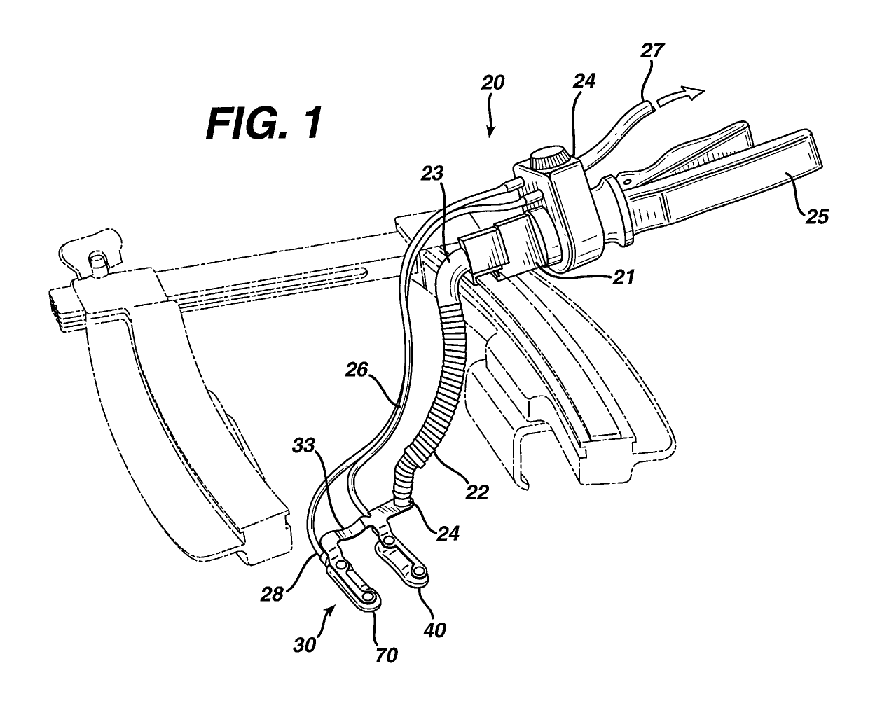

Referring to the figures, FIG. 1 illustrates a first embodiment of a stabilizer 20 for stabilizing the surface of the heart during a surgical procedure. Stabilizer 20 includes a mounting assembly 21 for connecting stabilizer 20 to a retractor rail (shown in shadow lines), a shaft 22 having a proximal end 23 connected to mounting assembly 21, and a distal end 24, and an actuator 25 connected to mounting assembly 21 for locking shaft 22 in a preferred position and/or locking mounting assembly 21 at a preferred position relative to the operative site. While stabilizer 20 is shown as having an articulating shaft 22, it may be coupled to a rigid shaft or arm, which may be malleable, articulating or hinged. Actuator 25 is preferably a manually actuated lever or trigger, movable from a natural position to an actuated position upon actuation by the user, but may be any other type of actuator known to those skilled in the art.

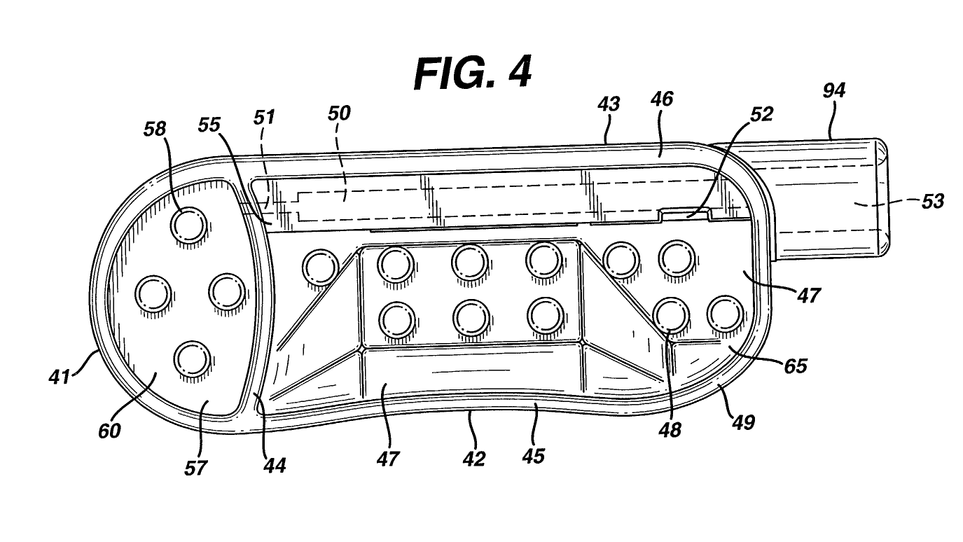

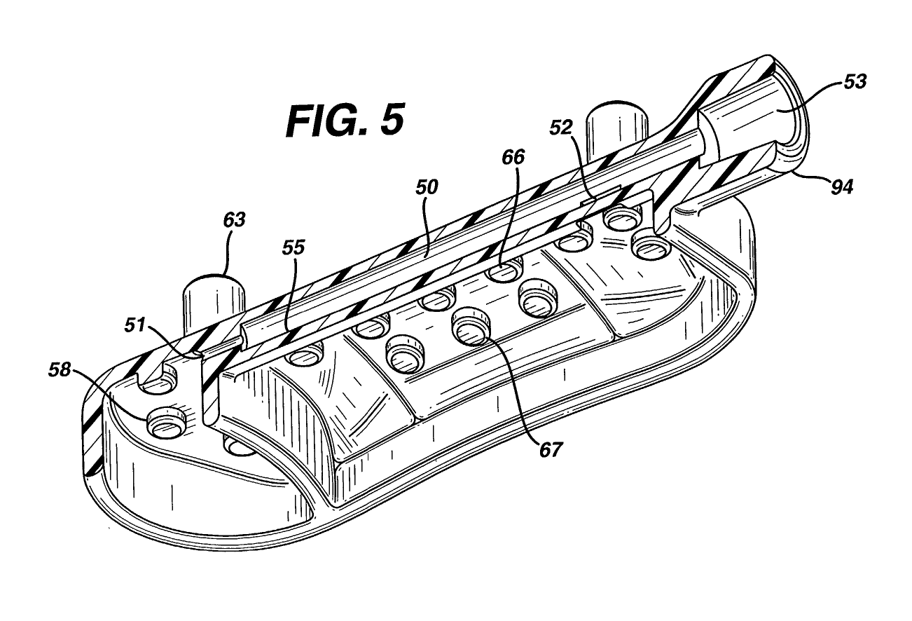

Stabilizer 20 further includes a suction assembly 30 configured to engage, for example, the surface of the heart on opposing sides of an anastomosis site. Suction assembly 30 includes a frame 33, and a first suction foot 40 and a second suction foot 70, connected one to the other by frame 33. First suction foot 40 and second suction foot 70 each include a stem 94 that extends from one end. Stem 94 includes an inlet port 53 (FIGS. 4 and 5) that fluidly communicates with a suction source. Inlet port 53 of both feet 40 and 70 are attachable to suction tubing 26 at a distal end 28 of the tubing. The proximal end 27 of suction tubing 26 is attachable to a suction source (not shown). As a result, when distal end 28 of suction tubing 26 is attached to inlet port 53, the suction source is in fluid communication with suction feet 40, 70. Generally, the suction available in a surgical setting ranges from 200 to 600 mmHg. Preferably, the suction source should provide a vacuum of approximately 400 mmHg. Suction tubing 26 may be connected to a valve 29 molded with, or connected to, actuator 25, that can be used to permit or prevent fluid communication between the suction source and suction tubing 26.

Stabilizer 20 may be mountable to a retractor, shown in shadow, an example of which is described in copending U.S. patent application Ser. No. 09/492,558, which is incorporated herein by reference, or to a surgical stand affixed to the operating table or on the operating room floor (not shown). Various accessory components may also be coupled to the rails of a retractor, including heart retractors and manipulators, CO₂ blowers, irrigators, suction devices, vascular clamps, lighting devices, catheters, and other devices.

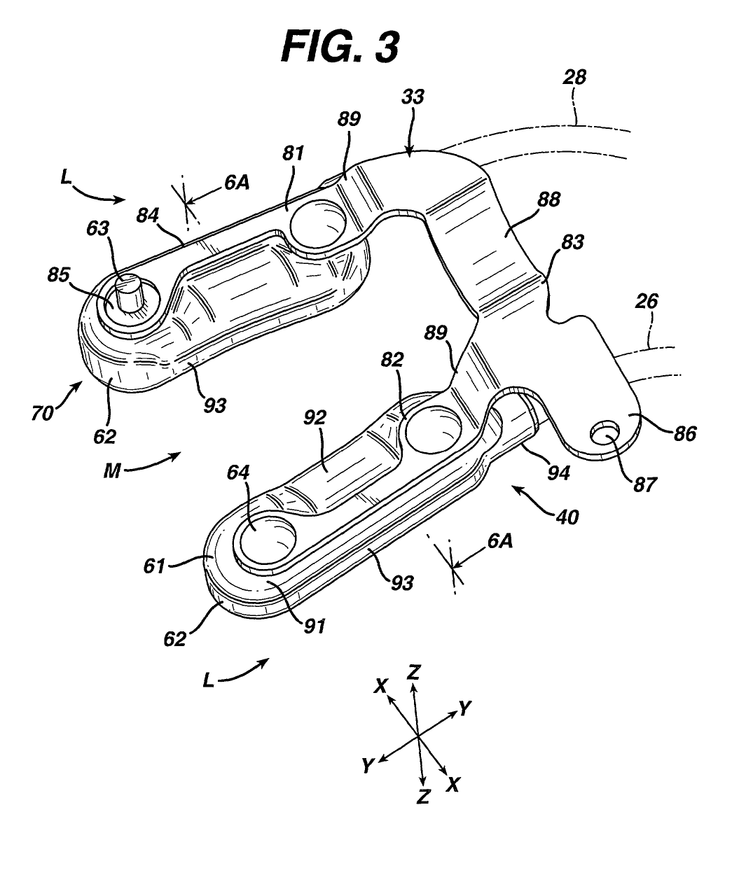

Referring to FIG. 3, frame 33 of suction assembly 30 is preferably substantially u-shaped and formed from 304 stainless steel. Frame 33 includes a first arm 81 and a second arm 82, each extending at approximately a right angle from either end of a cross member 83. Arms 81 and 82 each have a narrowed section 84 and a hole 85 on either side of narrowed section 84. Arms 81 and 82 are configured such that they are angled slightly away from one another, but generally parallel, and are spaced apart by a distance in the range of about 1-5 cm. Cross member 83 includes a projection 86 that includes a stem (not shown) that is seated within hole 87. Frame 33 is preferably pivotably mounted at the stem to distal end 24 of shaft 22 by an articulating joint (e.g., a ball joint) that may be locked and unlocked by means of actuator 25. This arrangement permits the feet to be positioned at various orientations relative to the shaft according to the angle of approach the practitioner uses to position stabilizer 20 within the opening into the chest cavity and relative to the location of the anastomosis site on the heart.

Frame 33 preferably includes three living hinges to permit frame 33 to be bent to conform to the anastomotic site. A living hinge 88 is located at approximately the midpoint of cross member 83, between arms 81 and 82, and a pair of living hinges 89 are located, one each, at the junction of arms 81 and 82 and cross member 83. Referring to the XYZ axes depicted in FIG. 3, living hinge 88 permits the arms 81 and 82 of frame 33 and hence suction feet 40 and 70 to be rotated in the XY plane (about the Z-axis) and in the XZ plane (about the Y-axis). Thus, feet 40 and 70 can be moved together or spread apart relative to one another in the XY plane, and rotated toward one another or away from one another in the XZ plane. Further, living hinges 89 permit suction feet 40 and 70 to be rotated in the YZ plane (about the X axis), that is up and down relative to cross member 83. In this way, the user may bend frame 33 to conform to convex or concave surfaces or certain saddle geometries, all of which exist on a heart surface and may be encountered at an anastomosis site.

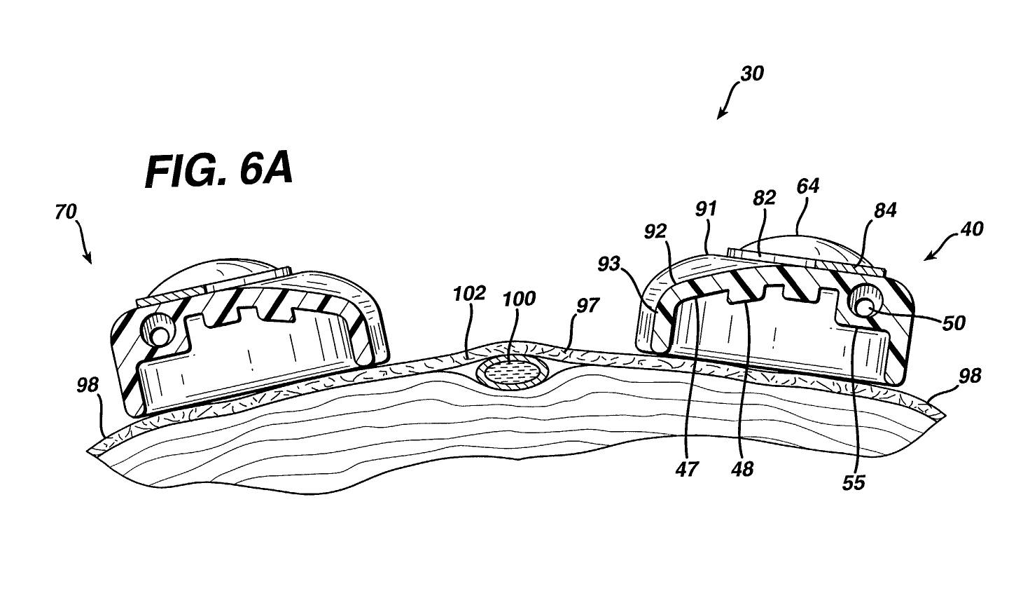

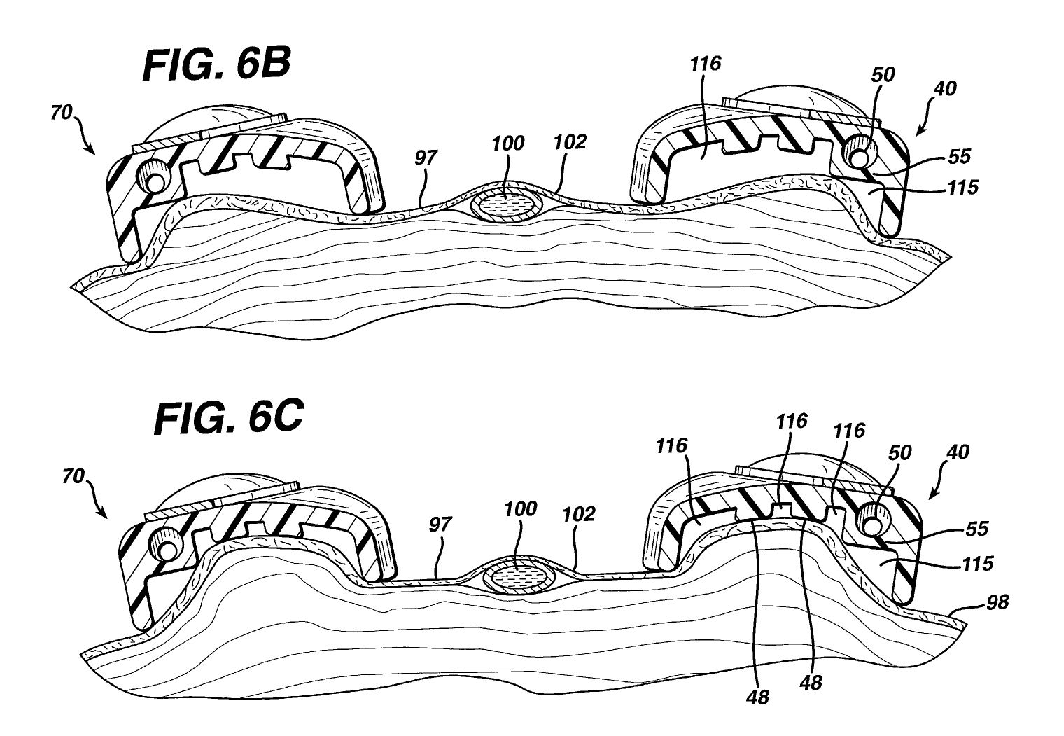

As discussed above, suction assembly 30 is configured to engage the surface of the heart on opposing sides of an anastomosis site, and preferably has a first suction foot 40 and a second suction foot 70, connected to one another by a frame 33. Suction feet 40, 70 are configured such that they are angled slightly away from one another, but generally parallel, and are spaced apart by a distance in the range of about 1-5 cm. The tissue located between feet 40 and 70 when the feet are positioned on the heart is referred to as the medial tissue, the location of which is indicated by arrow M in FIG. 3 and reference numeral 97 in FIG. 6A. The space outside between feet 40 and 70 when the feet are positioned on the heart is referred to as the lateral tissue, the location of which is indicated by arrow L in FIG. 3 and reference numeral 98 in FIG. 6A. That portion of suction feet 40 and 70 that lies closer to medial tissue 97 is referred to as the medial side; that portion of suction feet 40 and 70 that lies closer to lateral tissue 98 is referred to as the lateral side.

Suction assembly 30 is shown as a pair of generally parallel suction feet 40, 70, but may take the form of any number of shapes known in the art. For example, suction assembly 30 may consist of a single suction foot that takes the shape of a circle, an oval, semi-oval or a linear member. Other features and configurations may also be provided, such as those described in U.S. Pat. No. 5,807,243, assigned to the assignee of the present application and hereby incorporated herein by reference.

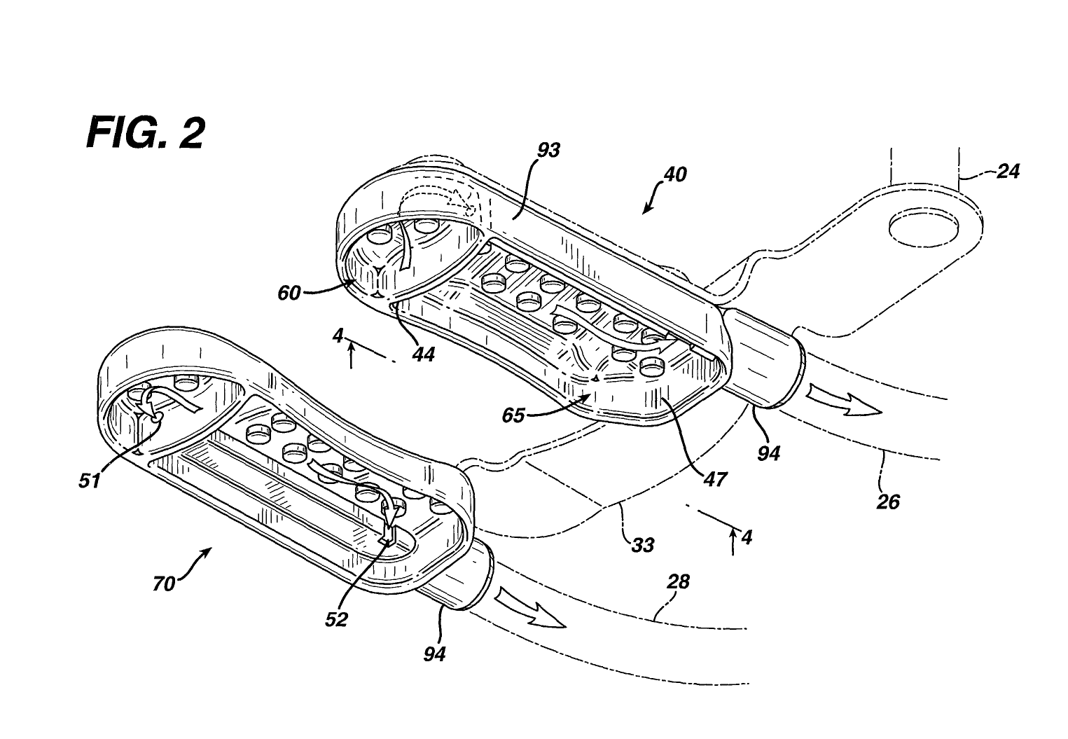

In one embodiment of the invention, suction feet 40 and 70 are mirror images of one another, with suction foot 40 shown in the figures as a left suction foot and suction foot 70 shown as a right suction foot. In describing the suction feet 40, 70, suction foot 40 will be described in detail. For the purpose of brevity, suction foot 70 will not be separately described, however for purposes of illustration, some elements described in connection with suction foot 40 are labeled in FIGS. 2 and 3 as being part of suction foot 70. It is understood that suction foot 70 preferably has components similar to those of suction foot 40.

Referring to FIG. 3, suction foot 40 is preferably formed as a housing 62, having an outer surface 61. Outer surface 61 includes a planar surface 91, formed to provide a base upon which arms 81 and 82 of frame 33 are seated, an outer wall 93 that extends around the perimeter of suction foot 40 and is connected to planar surface 91 on the lateral side, and an tapered surface 92 that extends from planar surface 91 to outer wall 93 on the medial side. Tapered surface 92 provides the user with additional room on the medial side of the suction feet for easier tool access to the medial tissue and the anastomosis site. In a preferred embodiment, the height of outer wall 93 varies from a range of 0.39 cm to 0.55 cm, but most preferably about 0.47 cm, on the lateral side to a range of 0.32 cm to 0.48 cm, but most preferably about 0.40 cm, on the medial side.

Referring to FIGS. 2 and 4, housing 62 also includes an inner surface 47 that shares a perimeter edge 49 with outer wall 93. Perimeter edge 49 includes a medial edge 45 located on the medial side of foot 40 and a lateral edge 46 located on the lateral side of foot 40. Housing 62 is divided into a distal chamber 60 and a proximal chamber 65 by a dividing wall 44 that extends away from inner surface 47 and substantially parallel to outer wall 93. In a preferred embodiment, distal chamber 60 has a volume that is less than the volume of proximal chamber 65. While suction foot 40 is shown with two chambers, one skilled in the art will appreciate that housing 62 may be divided into additional chambers, depending on how the suction foot would be used. Preferably, where a suction foot includes more than two chambers, at least one of the chambers would have a different volume as compared to the other chambers.

Referring to FIGS. 4 and 5, housing 62 also includes proximal pegs 48 and distal pegs 58 that extend away from inner surface 47 and substantially parallel to outer wall 93. The pegs 48, 58 are preferably cylindrical and parallel to one another as shown in this embodiment, but may vary in size, spacing, and orientation. The tips of pegs 48, 58 may have a flat surface 66 or an angled surface 67. Pegs 48, 58 are spaced sufficiently distant from perimeter edge 49 to allow the organ surface to seal properly against the left and right feet 69, 68. In addition, the pegs create a tortuous path for any particulate and therefore also act as a filter. The length of pegs 48, 58 and the depth of at least one chamber, in this case proximal chamber 65, are configured to ensure that a vacuum manifold is maintained above the captured tissue during stabilization by preventing epicardial fat or other compliant tissue from contacting inner surface 47, while at the same time permitting as much tissue to be captured within proximal chamber 65. In a preferred embodiment, the diameter of pegs 48, 58 is approximately 24 mm, the height is approximately 8 mm, and the height of outer wall 93 is approximately 48 mm. As such the ratio between the height of outer wall 93 and the peg height is approximately 6:1. The distance between pegs 48 in proximal chamber 65 along the width of the chamber is approximately 20 mm, and along the length of the chamber is approximately 32 mm. The distance between pegs 58 in distal chamber 60 along the width of the chamber is approximately 48 mm, and along the length of the chamber is approximately 24 mm.

Housing 62 also includes a shoulder 55 molded into an inner surface 47 near the lateral side of foot 40 along the length of proximal chamber 65. Shoulder 55 includes a manifold 50 that fluidly communicates with inlet port 53 of stem 94, which in turn communicates with a suction source. Shoulder 55 also has a distal orifice 51 that provides fluid communication between manifold 50 and distal chamber 60, and a proximal orifice 52 that provides fluid communication between manifold 50 and proximal chamber 65. Proximal and distal orifices 50 and 52, the cross section of manifold 50, and the volume of each of the chambers 60, 65 are configured such that chambers 60 and 65 have vacuum independence. For purposes of this application “vacuum independence” means that the vacuum produced in each of the chambers or each of the feet, is such that, if suction is lost in one chamber or one foot such that the chamber or foot no longer holds cardiac tissue, the other chamber or foot has sufficient vacuum to maintain hold of cardiac tissue. In other words, the loss of vacuum in one chamber or foot does not cause the remaining chamber or foot to also lose its hold on cardiac tissue. Thus, in a preferred embodiment, because the distal orifice 51, proximal orifice 52 and the volumes of chambers 60, 65 are sized appropriately, in the event suction is lost in one foot, the resulting pressure drop does not cause the remaining foot to lose the vacuum needed to maintain a hold on the cardiac tissue captured previously. Thus, suction foot 40 has vacuum independence from suction foot 70.

In a preferred embodiment, the cross section of manifold 50 ranges from 0.020 cm² to 0.030 cm², but most preferably is approximately 0.025 cm², the area of distal orifice 51 ranges from 0.0036 cm² to 0.0056 cm², but most preferably is approximately 0.0046 cm², and the area of proximal orifice 52 ranges from 0.0062 cm² to 0.0082 cm², but most preferably is approximately 0.0072 cm². The preferred ratio of distal orifice to proximal orifice to manifold cross section is approximately 1:1.6:5.3. The volume defined by inner surface 47 and dividing wall 44 of proximal chamber 65 ranges from 0.268 cm³ to 0.402 cm³, but most preferably is approximately 0.335 cm³, and the volume defined by inner surface 47 and dividing wall 44 of distal chamber 60 ranges from 0.09842 cm³ to 0.1030 cm³, but most preferably is approximately 0.0936 cm³. The ratio of the distal chamber volume to the proximal chamber volume is preferably approximately 1:3.6.

Suction feet 40, 70 are preferably injection molded using a polycarbonate or other suitable plastic, although any other manufacturing method or materials known to one skilled in the art may be used. The most preferred housing material is Dow Makrolon, as it is capable of bending to conform to frame 33. Heat stakes 63 extend upwardly from planar surface 91 (see FIG. 6A) of housing 62 at two locations so as to mate with holes 85 of frame 33. Heat stake 63 is shown in its original configuration extending upwardly from suction foot 70 prior to be heated to its final configuration identified as heat stake 64 on suction foot 40. In this way, suction feet 40 and 70 are each connected to frame 33 at two locations along their length. Alternatively, frame 33 may be overmolded to attach suction feet 40 and 70 to frame 33.

Referring to FIG. 6A, in another feature of the invention, suction feet 40 and 70 can be designed to pull taut medial tissue 97 to better expose a coronary or target artery 100 for purposes of performing CABG. Medial tissue, depicted as reference numeral 97, is that tissue located between the first foot and the second foot once the suction assembly has been positioned on the heart. Medial edge 45 of suction feet 40, 70 can be configured to preferentially permit medial tissue to be drawn or pulled into at least one chamber. Preferably the chamber having the larger volume is configured to draw medial tissue into it, and in the depicted embodiment that chamber is proximal chamber 65. Referring to FIG. 8, to create an edge that permits medial tissue 97 to move relative to the edge, medial edge 45 of proximal chamber 65 includes a means 105 to reduce friction relative to the medial tissue. The friction-reducing means 105 includes rounding or chamfering medial edge 45 or the application of a hydrophilic coating to medial edge 45 or covering medial edge 45 with a friction-reducing material or modifying the surface of medial edge 45 to make it smoother, such as by a plasma treatment, or any combination of any of the foregoing.

In addition, to enhance the preferential drawing medial tissue 97, lateral edge 46 of at least one of the chambers 60, 65 can include a means 110 to increase friction relative to the lateral tissue to resist lateral tissue from being drawn into the chambers. The friction-increasing means 110 includes squaring off the lateral edge, or creating a textured, knurled, roughened, or covered lateral or coating the lateral edge with a friction-enhancing material or any combination of any of the foregoing. In this way, lateral edge 46 of proximal chamber 65 may include friction-increasing means 110 so that more tissue on medial side 45 of each foot is drawn into proximal chamber 65 than on the lateral side, thereby causing the medial tissue to be pulled taut to better expose target artery 100 for CABG.

To further enhance the preferential draw of medial tissue, shoulder 55 is molded closer to lateral edge 46 than medial edge 45 such that when lateral tissue 98 has been pulled into the chamber, lateral tissue 98 contacts shoulder 55 thereby providing further resistance to tissue flow from the lateral side. Shoulder 55, in effect, increases the friction encountered by lateral tissue 98, as the tissue is subject to two contact points on the lateral side, lateral edge 46 and shoulder 55, as compared to the one contact point on the medial side, medial edge 45. Further, once lateral tissue 98 contacts shoulder 55, it isolates the region above lateral tissue 55 from a vacuum source thereby reducing the area of lateral tissue 98 acted upon by the vacuum within chambers 60, 65.

When it is time to perform the coronary anastomosis, mounting base 21 of stabilizer 20 is positioned along one the rails or on a crossbeam of a retractor (shown in shadow lines in FIG. 1) at the desired position. Suction assembly 30 is then positioned so that the distal chamber 60 of suction foot 40 engages the epicardium near the anastomosis site. Typically, suction feet 40 and 70 are positioned on opposing sides of the target coronary artery, aligned with the anastomosis site. Alternatively, suction foot 40 may be positioned so as to engage the coronary artery itself upstream of the anastomosis site to occlude the coronary artery to provide hemostasis during the anastomosis.

With reference to FIG. 6A, one method of using stabilizer 20 is depicted. Suction feet 40 and 70 are positioned such that they contact the epicardial tissue, in this case on opposing sides of the target artery 100. Suction assembly 30 is placed in contact with the fascia layer 102 of the epicardial tissue. The user may then lock suction assembly 30 by actuating actuator 25, which locks shaft 22 relative to mounting assembly 21 and relative to suction assembly 30. Suction is then activated and distal chamber 60 is evacuated prior to proximal chamber 65 due to the smaller volume of distal chamber 60. The evacuation of chambers 60 and 65 is shown in FIG. 2 by arrows, which indicate that the air within chambers 60 and 65 is drawn out via distal orifice 51 and proximal orifice 52, respectively. As a result, distal chamber 60 captures tissue first. In the event the practitioner needs to adjust the position of the suction feet, the practitioner may still move the proximal portion of suction foot 40 relative to the distal portion of suction foot 40.

Turning to FIG. 6B, tissue is drawn into feet 40 and 70 once a sufficient vacuum exists. Medial tissue is preferentially pulled into the medial side of suction foot 40 due to the friction reduction means. As medial tissue 97 is drawn into the feet, fascia 102 begins to thin and target artery 100 is more prominently displayed. Tissue is drawn into chamber 65 under the vacuum until it contacts shoulder 55, as shown in FIG. 6B. At this point, region 115 below shoulder 55 and between lateral tissue 98 is isolated from any vacuum and thus vacuum acts only on the tissue located below the area shown as 116. Area 116 is in fluid communication with manifold 65 via proximal orifice 52 (see FIG. 2). The vacuum in area 116 continues to draw tissue into chamber 65 until tissue contacts pegs 48. At this point, tissue is firmly captured within suction feet 40 and 70, the relative movement of the heart in the area of medial tissue 97 is minimized, and target vessel 100 is exposed such that the practitioner is better able to perform a surgical procedure, such as CABG. Finally, the heart continues to beat and the remainder of the heart tissue outside the area of the surgical procedure is permitted to move unimpeded.

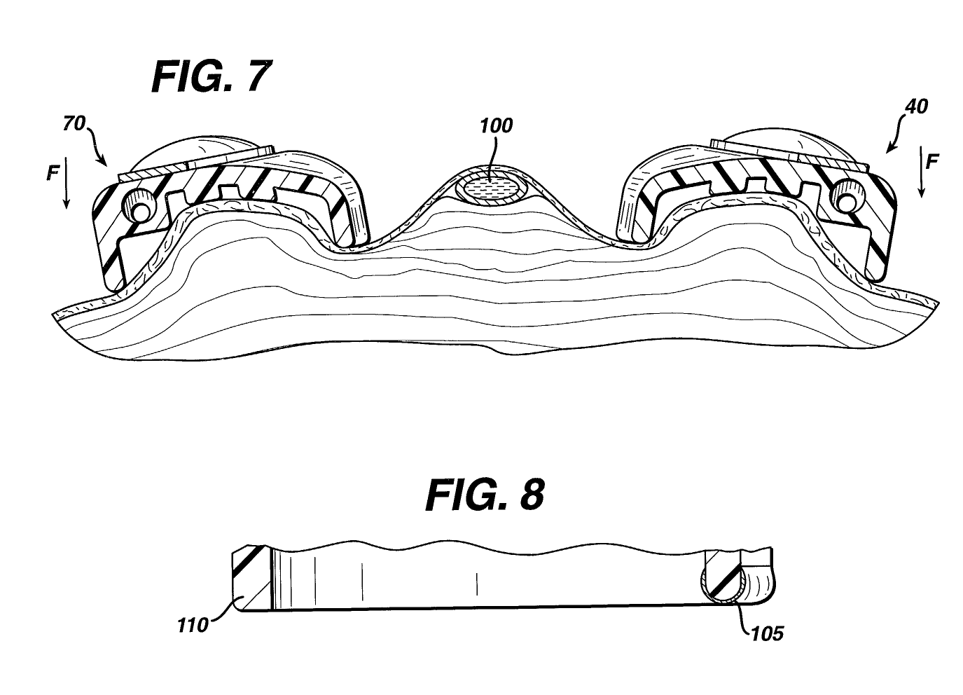

With reference to FIG. 7, an alternate method of using suction assembly 30 is depicted. Rather than placing suction assembly 30 on the epicardial surface, the user applies a mechanical force F on the epicardial tissue prior to initiating suction. Similar to that method depicted in FIGS. 6A-6C, epicardial tissue is drawn into suction feet 40 and 70 until it contacts pegs 47. Applying the mechanical force may cause target vessel 100 to more prominently exposed than in the absence of such a force.

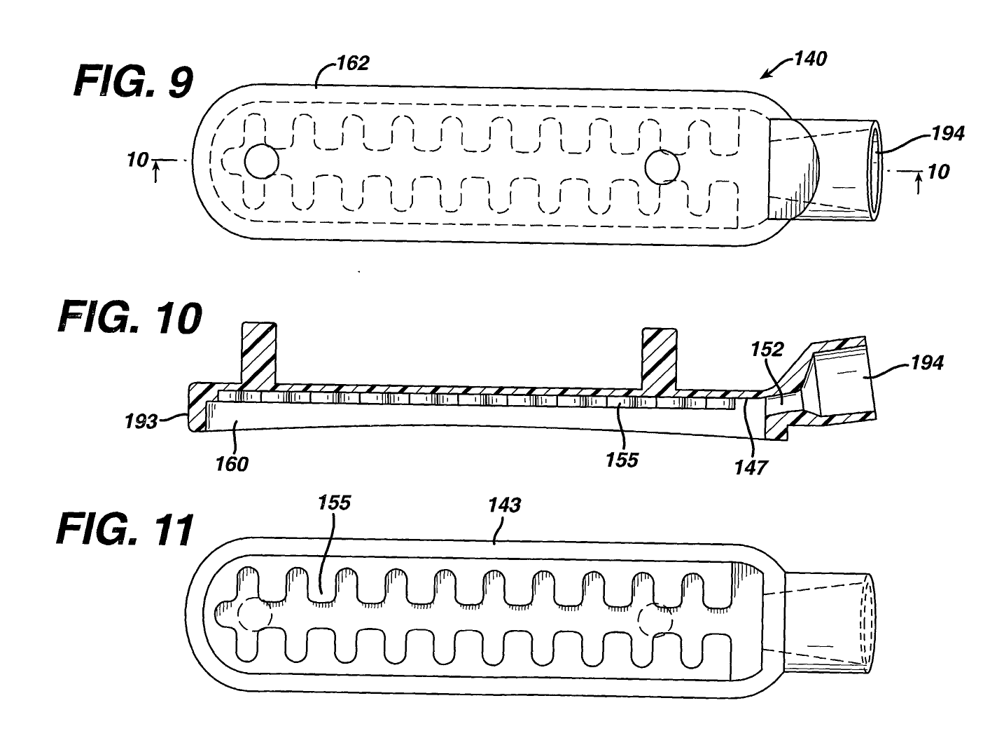

A second embodiment of the invention is depicted at FIGS. 9-11. A suction foot 140 is depicted having a housing 162 that includes an inner surface 147 and a wall 193. Suction foot 140 can be configured to have many of the same features of suction feet 60, 65 described above, including vacuum independence and preferential draw of medial tissue. Suction foot 140 differs from suction feet 60, 65 in that it has a single chamber 160 that communicates with a suction source (not shown) via an orifice 152 and an inlet port 194. A shoulder 155 is molded about the outer perimeter of an inner surface 147 of chamber 160 in a serpentine fashion to prevent tissue that has been drawn into chamber 160 from blocking orifice 152 so as to maintain tissue capture by maintaining suction above a sufficient area of tissue.

While the above is a complete description of the preferred embodiments of the invention, it will be appreciated that various equivalents, modifications, additions and substitutions may be made without departing from the scope thereof. Therefore, the above should not be taken as limiting the scope of the invention, which is defined by the following claims. For example, any foot described herein may be used with any arm or shaft described herein. Furthermore, although the retractor is designed for use with a median stemotomy, the stabilizer 20 may be used with retractors in other parts of the chest and specifically with smaller, less-invasive openings, without departing from the scope of the invention. Finally, while suction assembly 30 has the ability to provide suction stabilization, it may also be used for simple mechanical stabilization as well. Furthermore, the invention provides various independent aspects and is not limited to a single indispensable feature, advantage or aspect. Thus, each feature or aspect of the invention may be considered independent of the other features, advantages and aspects of the present invention.

What is claimed is:

1. A suction foot for stabilizing an epicardial surface of the heart, comprising:
a housing having a first chamber that defines a first volume and a second chamber that defines a second volume, and wherein the first volume is less than the second volume; and
the housing having a manifold that fluidly communicates with the first chamber through a first orifice and fluidly communicates with the second chamber through a second orifice.

2. The suction foot of claim 1, wherein at least one of the first chamber and second chamber includes a shoulder comprising the manifold.

3. The suction foot of claim 2, wherein the shoulder is molded as part of the second chamber.

4. The suction foot of claim 1, wherein the manifold has a cross section, and the cross section, the first orifice, the second orifice, the first volume and the second volume are configured such that the first chamber is vacuum independent from the second chamber.

5. A stabilizer for stabilizing an epicardial surface of the heart, comprising:
a bendable frame;
a first suction foot connected to the frame;
a second suction foot connected to the frame at a location spaced apart from the first suction foot; the frame including at least two living hinges to permit the frame to be bent to a position that conforms to the epicardial surface of the heart.

6. The stabilizer of claim 5, wherein the frame comprises a cross member, a first arm connected to the cross member, a second arm connected to the cross member at a location spaced apart from the first arm, and wherein the first suction foot is connected to the first arm and the second suction foot is connected to the second arm.

7. The stabilizer of claim 6, wherein the frame includes a first living hinge located along the length of the cross member between the first arm and the second arm, a second living hinge located at the junction of the first arm and the cross member, and a third living hinge located at the junction of the second arm and the cross member.

8. The stabilizer of claim 6, wherein first arm has two spaced-apart holes and the first suction foot includes a pair of heat stakes that project upwardly from the top surface of the first suction foot at spaced-apart locations to mate with one each of the two holes, and wherein the first arm is connected to the first suction foot by melting the heat stakes.

9. The stabilizer of claim 6, through the includes a distal chamber and a proximal chamber, and comprising a suction source connected to at least one of the first suction foot and the second suction foot.

10. A stabilizer for stabilizing an epicardial surface of the heart comprising:
an arm; and
a first foot having at least one suction chamber and including a medial edge having a friction-reducing means to promote medial tissue being drawn into the suction chamber.

11. The stabilizer of claim 10, wherein the first foot comprises a distal chamber and a proximal chamber, the proximal chamber having a larger volume than the distal chamber and comprising the medial edge having the friction-reducing means.

12. The stabilizer of claim 11, wherein the first foot includes a lateral edge having friction-increasing means to resist lateral tissue being drawn into the suction chamber.

13. The stabilizer of claim 10, wherein the friction-reducing means comprises a chamfered medial edge.

14. The stabilizer of claim 10, wherein the friction-reducing means comprises applying a hydrophilic coating to the medial edge.

15. The stabilizer of claim 10, wherein the friction-reducing means comprises modifying the medial edge surface to make it smoother by a plasma treatment.

16. A suction foot for stabilizing an epicardial surface of the heart comprising:
a housing including a chamber having an orifice for fluidly communicating with a suction source, the chamber having a wall and an inner surface; and
a shoulder connected to the perimeter of the inner surface adjacent the wall that is adapted to prevent tissue drawn into the chamber from blocking the orifice, while providing sufficient vacuum to maintain tissue capture.

17. A method of performing a coronary anastomosis on a heart of a patient comprising:
providing a stabilizer having a shaft and a suction assembly, the suction assembly having a first foot and a second foot spaced apart from the first foot, the first foot and second foot having at least one chamber, and an inlet port that fluidly communicates with a suction source and the at least one chamber, and wherein the chamber includes a medial edge that includes a friction-reducing means;
accessing a coronary artery on the patient's heart;
positioning the first foot and second foot to contact the surface of the heart so that the coronary artery is positioned between the first foot and the second foot;
applying suction to the chamber of the first foot and the second foot via the suction source to evacuate the chamber;
preferentially drawing medial tissue into the chamber; and
performing an anastomosis on the coronary artery.
